(19) United States
(12) Patent Application Publication
(10) Pub. No.: US 2020/0214580 A1
(43) Pub. Date: Jul. 9, 2020

(54) Catheters and Apparatuses for Combined Imaging and Pressure Measurement

(71) Applicants: Wei Kang, Somerville, MA (US); Chenyang Xu, Devens, MA (US)

(72) Inventors: Wei Kang, Somerville, MA (US); Chenyang Xu, Devens, MA (US)

(73) Assignee: KOTL LLC, Devens, MA (US)

(21) Appl. No.: 16/238,945

(22) Filed: Jan. 3, 2019

(51) Int. Cl. A61B 5/0215; A61B 8/04; A61B 8/12; A61B 8/08; A61B 5/00; A61B 5/1459

(52) U.S. Cl. CPC A61B 5/02154; A61B 8/04; A61B 8/12; A61B 5/1459; A61B 5/6852; A61B 5/0066; A61B 8/5215

(57) ABSTRACT

The invention relates to catheters and apparatuses for combined imaging and obtaining pressure measurement in a vessel having a stenosis. The imaging can be an optical imaging technology or ultrasound imaging. The apparatus can include a combined probe comprising an imaging channel and a pressure measurement channel, and a signal processor in communication with the imaging channel and the pressure measurement channel.

### BACKGROUND OF THE INVENTION

In the field of blood vessel treatment, the severity of a stenotic lesion can be assessed by means of structural imaging and/or measurement of physiological parameters. For instance, Optical coherence tomography (OCT) and intravascular ultrasound (IVUS) are examples of imaging methods for revealing the blood vessel microstructure. They are used for determining vessel lumen size, stent deployment and other clinically relevant information. The image acquisition is accomplished by utilizing a minimally invasive catheter with distal miniature optical or ultrasound assembly. Meanwhile, blood pressure has long been a clinically significant physiological parameter. Fractional flow reserve (FFR), for instance, is a well-accepted measurement method to evaluate lesion severity in situ. It utilizes a fine-wire or a probe with pressure transducer mounted near the distal tip, which can be inserted into the blood vessel for precise blood pressure measurement. Traditionally, FFR wire or probe employs electronic pressure transducer. Alternatively, optical pressure transducers have been used in recent years, demonstrating improved performance on noise suppression and drift immunity. To use these optical transducers, a fiber-optic based wire or catheter is inserted. Combining OCT or other imaging modalities with pressure measurement into one device addresses the need for both structural and functional information of the blood vessel and is technically plausible.

There has been effort to combine OCT imaging and the electrical/optical transducer based FFR measurement into one device. A straight forward approach is to place the OCT channel and the FFR channel side-by-side, each in its own protective sheath. However, side-by-side arrangement results in an undesirable larger crossing profile.

To achieve a smaller crossing profile in the case of the optical pressure transducer, a more compact approach is to use the same optical fiber for both the OCT and FFR. As a coherence imaging method, OCT typically uses single mode fiber. On the other hand, for FFR, apparatuses and methods have been proposed to use the same single mode fiber as OCT for delivering and collecting light. This approach requires complicated structures such as fiber tip beam splitters that are costly and difficult to manufacture.

Accordingly, catheters and apparatuses are needed for improving the imaging and pressure measurement combination device with smaller crossing profile, lower manufacturing cost and improved performance.

### FIELD OF THE INVENTION

The present invention is in part in the field of fiber-optic systems for intravascular imaging and pressure measurement.

### BACKGROUND ART

US Pat. Publ. No. 2014/0094697 by Christopher Petroff, et al. (“Petroff”), describes current equipment and methods for treating blood vessels with stenotic lesions and other full or partial blockages. U.S. Pat. No. 8,478,384 to Joseph M. Schmitt, et al. (“Schmitt”), describes a combined OCT/pressure measurement probe.

US Pat. Publ. No. 2017/0188834 by Weina Lu, et al. (“Lu”), describes multiple configurations of combined OCT/pressure measurement probes.

### SUMMARY OF THE INVENTION

In part, the invention relates to catheters and apparatuses for both imaging and obtaining pressure measurement in a vessel having a stenosis. The imaging can be an optical imaging technology or ultrasound imaging. In one exemplary embodiment, the apparatus includes a combined probe comprising an imaging channel and a pressure measurement channel, and a signal processor in communication with the imaging channel and the pressure measurement channel.

In one aspect, the invention in one exemplary embodiment relates a probe that includes at least a bore in a body, wherein the body has at least one opening to environment allowing environment pressure transmitted to the bore. The probe can have an optical imaging channel which has a first optical fiber located in the bore transmitting the light for optical imaging, an optical lens inside the bore and in communication with the first optical fiber. The optical imaging channel can be rotated about the longitudinal axis of the bore. The probe can also include a pressure measurement channel which does not rotate when acquiring signal. In one exemplary embodiment, the pressure measurement channel can include a second optical fiber located in the bore transmitting the light for pressure measurement and an optical pressure transducer inside the bore and in communication with the second optical fiber. In another exemplary embodiment, the probe can include a pressure measurement channel which includes conductive wires located in the bore transmitting the electrical signal for pressure measurement and an electrical pressure transducer inside the bore and in communication with the conductive wires. The probe can include a torque transmission coil that rotates inside the bore. The optical imaging channel and the pressure measurement channel both partially resides inside the torque transmission coil.

In another exemplary embodiment, the imaging channel can be configured to perform ultrasound imaging instead of optical imaging. The ultrasound imaging channel can have an ultrasound transducer and electrical wires located in the said bore in communication with the transducer transmitting electrical signals. The ultrasound imaging channel can be rotated about the longitudinal axis of the said bore.

In another aspect, the invention relates to a probe comprising a first end and a second end. The first end, defined as the distal end, is positioned in the location of interest in the blood vessel where the imaging and the pressure measurement are acquired. The second end, defined as the proximal end, includes a mating unit that provides connections with a system to process the imaging signal and the pressure signal.

In yet another aspect, the invention relates to a signal processor defined as a combined imaging/pressure measurement engine. The engine, in communication with the combined probe, provides the light that is required to generate the imaging and pressure measurement signals, and received the signals for further processing.

### BRIEF DESCRIPTION OF THE DRAWINGS

FIG. 1 shows an overall diagram of the combined imaging and pressure measurement probe.

FIG. 2 shows a side cut-view diagram of an exemplary embodiment of the distal end of the probe.

FIG. 3 shows the cross-sectional view diagram of an exemplary embodiment of the probe body.

FIGS. 4-6 shows other side cut-view diagrams of exemplary embodiments of the distal end of the probe.

FIG. 7 shows a side cut-view diagram of an exemplary embodiment of the proximal mating unit.

FIG. 8 shows another side cut-view diagram of an exemplary embodiment of the proximal mating unit.

FIG. 9 shows an end-view diagram of another exemplary embodiment of the proximal mating unit.

FIG. 10 shows another side cut-view diagram of an exemplary embodiment of the proximal mating unit.

FIG. 11 shows an exemplary embodiment of the system that can be used with the combination probe.

### DETAILED DESCRIPTION OF THE INVENTION

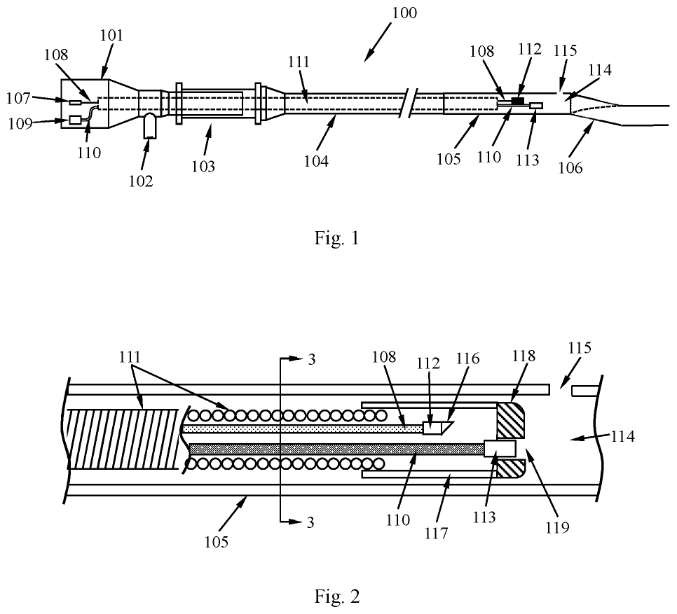

Referring now to the invention in more detail, FIG. 1 shows the overall view diagram of an exemplary optical imaging and pressure measurement combination probe 100. The probe takes form of a catheter for easy insertion into blood vessels. FIG. 2 shows the magnified cut-view diagram of the distal end of one exemplary embodiment. The probe's outside housing is comprised of a proximal mating unit 101, a liquid purge port 102, a telescoping section 103, a proximal sheath 104, a distal sheath 105, and a rapid-exchange section 106. Inside the housing there are rotary inner parts including a first optical connector 107, a first optical fiber 108, a second optical connectors 109, a second optical fiber 110, a torque transmission coil 111, an optical lens assembly 112, and an optical pressure sensing transducer 113. The probe can be placed inside a blood vessel which could be filled with blood or other liquid. The probe sheath defining a bore 114, where the sheath has at least one opening 115 to the environment allowing the environmental pressure transmitted to the bore. The bore 114 can have a longitudinal axis. The optical fiber 108 located in the bore is in communication with an optical lens assembly 112 inside the bore. In some embodiments, there is also a beam steering component with a reflective surface 116 for directing the light from the fiber 108 to a substantial angle with respect to the longitudinal axis of the bore 114. In FIGS. 1-6, an exemplary single-layer torque coil 111 is used for to illustrating the general rotatable structure for transmitting torque force. However, multiple-layer torque coils or other torque-transmission device can also be used.

In one exemplary embodiment, an optical pressure transducer 113 inside the bore is in communication with the optical fiber 110. In another exemplary embodiment, the transducer 113 can be an electrical pressure transducer, which is in communication with a plurality of electrical wires 110. To illustrate the method without loss of generality, an optical transducer and an optical fiber is used in FIGS. 1-5. The imaging fiber 108 and the pressure measurement fiber 110 are positioned to the distal end of the torque coil 111. When a beam steering component is used in the optical lens assembly 112, the imaging fiber 108 is positioned such that the pressure measurement fiber 110 is not in the path of the steered beam from reflective surface 116.

Referring to FIG. 2, in one exemplary embodiment, there can be an additional protective sheath 117 attached to the torque coil 111 to encompass the optical lens assembly 112 and the pressure transducer 113. This sheath can be a heat-shrinkable tubing, composed of a material such as PET or Teflon. The sheath is made of material of substantially small optical attenuation in the optical wavelength band used for the optical imaging. The shrinkable tubing can have an extended length to encompass a portion of the torque coil 111, such that when heat is applied, the contraction of the shrinkable tubing fixates to the torque coil, encompassing both the optical lens assembly 112 and the pressure transducer 113. In an additional exemplary embodiment, the tube 117 can be glued to the torque coil. The distal end of this protective sheath can have a round tip 118 to improve sliding along the bore 114. In one exemplary embodiment, there can be an opening 119 at the distal end so that the transducer 113 can be inserted to measure the pressure in the bore 114. In an additional exemplary embodiment, a low durometer silicone gel is inserted into the opening 119 between the pressure sensing surface of the transducer 113 and the bore 114. The gel preferably has low enough durometer to accurately transmit pressure to the sensing diaphragm of the transducer and not interfere with the diaphragm's motion when the gel and the diaphragm are in contact.

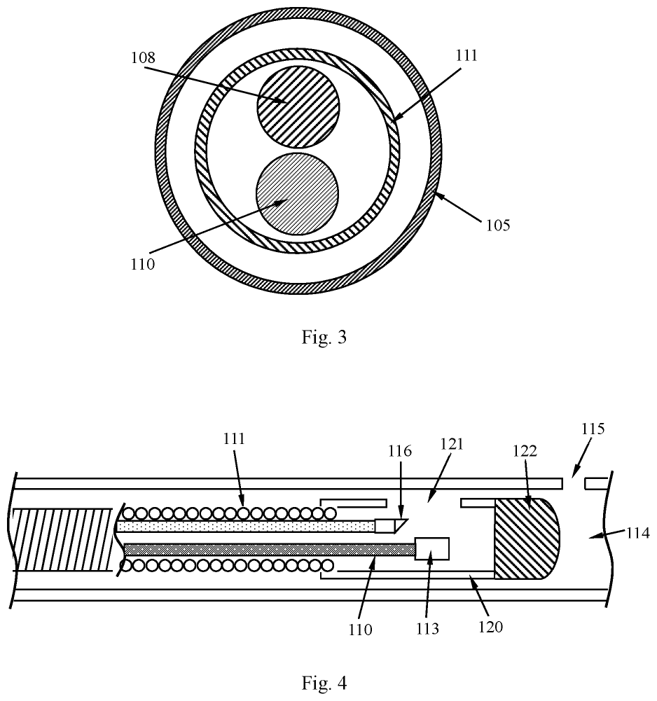

FIG. 3 shows the cross-sectional view diagram of the exemplary embodiment shown in FIG. 2 cut along the AA′. The torque coil 111 encompasses both the imaging fiber 108 and the pressure measurement fiber 110. The torque coil 111, the imaging fiber 108 and the pressure measurement fiber 110 can be rotated as one unit inside the protective sheath 105. This way, the imaging fiber and the pressure measurement signal are going through the same torque coil, the catheter crossing profile is reduced. It should be noted that the torque coil is an exemplary for torque transmission and by no means limiting the scope of this invention. There are other torque transmission methods known in the art, such as torque wire, torque rope, torque tubes, which are also within the scope of this invention. It should also be noted that the imaging channel may include other modalities such as ultrasound imaging, whose signal transmission is through electrical wires, and the pressure measurement channel may utilize electrical wires for communication with electrical pressure sensors. It should also be noted some signal transmission lines, such as optical fibers, are capable of torque transmission themselves and do not require extra torque transfer means. The variety of imaging modalities, the different torque transfer means, and the different imaging and pressure measurement signal transmission lines are all within the scope of this invention.

FIG. 4 shows the magnified cut-view diagram of the distal end of another exemplary embodiment. A protective sheath 120 can be made of metal or polymer, which is fixed to the torque coil by methods such as gluing or welding. 111. The sheath has an opening 121 that allows the pressure transmission such that pressure sensor 113 can measure the pressure in the bore 114. The opening 121 also allows the beam steered at the optical lens assembly 116 can exit the probe if the sheath 120 was made of non-transparent material such as metal. The distal end of this protective sheath can have a round tip 122 or springs to improve sliding along the bore 114.

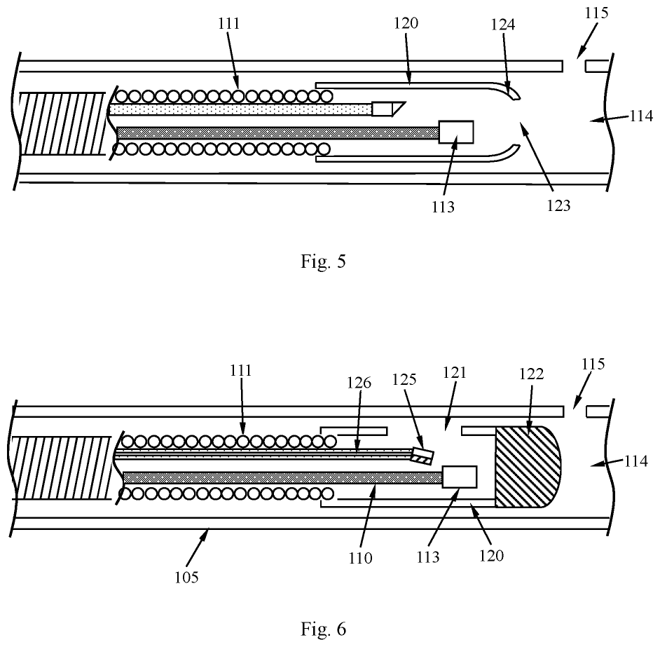

FIG. 5 shows the magnified cut-view diagram of the distal end of another exemplary embodiment. Compared to the embodiment in FIG. 4, the distal end of the protective sheath 120 has an opening 123 so that the pressure transducer 113 is in communication with the bore 114. A tapering structure or a chamfer 124 could be added to improve the sliding of the protective sheath 120 along the bore 114.

The optical imaging channel can be replaced by other imaging modalities such as ultrasound imaging. To illustrate the configuration without loss of generality, FIG. 6 shows an exemplary embodiment that is similar to FIG. 4 with the difference of an ultrasound imaging channel instead of an optical imaging channel. The ultrasound transducer 125 is made of piezoelectric crystal. It emits ultrasound to and receives echo from the blood vessel through the opening 121 of the protective sheath 120. Electrical wire pair 126 is connected to the transducer 125. It provides the alternating voltage necessary for ultrasound emission and transmits the electrical signal converted from the echo by the transducer. A low viscosity fluid such as saline or gel can be used to fill the space inside the sheath 120 and in the bore 114 in order to reduce the ultrasound reflection on the sheath 105.

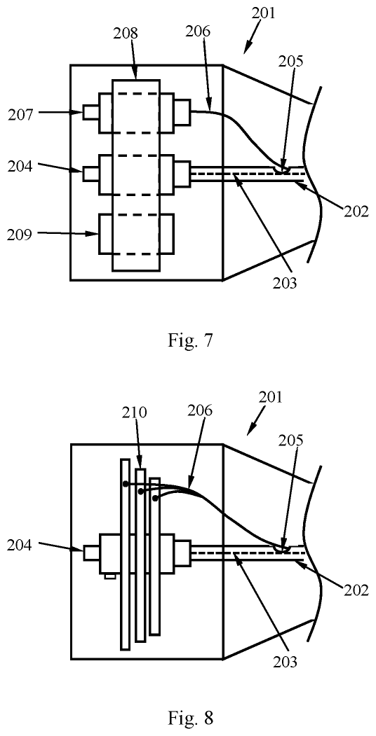

FIG. 7 shows an exemplary embodiment of the proximal mating unit 201, which includes a longitudinal tube 202, such as a metal hypotube. The tube can be fixated to the torque transmitting coil. A first optical fiber 203 for optical imaging can be positioned inside the tube and terminated by a first optical connector 204. The optical connector can also be fixated to the tube 202. When the optical imaging is performed, the optical connector 204, longitudinal tube 202, and the optical fiber 203 are rotated as one unit. In another exemplary embodiment, there can be a small opening on the tube 202, which allows for the exit of the pressure measurement channel. The pressure measurement channel can have either a second optical fiber or electrical wires 206, which is terminated by a second optical connector or an electrical connector 207, respectively. There can be an arrangement 208 such as a frame which fixates the two connectors 204 and 207 such that they can be rotated as one unit during optical imaging. There can also be counterbalancing weights 209 to balance the centrifugal forces during rotation.

FIG. 8 shows another exemplary embodiment of the proximal mating unit 201, which can be used for imaging and pressure measurement combination probes where one channel is optical while the other is electrical. One disadvantage in the assembly FIG. 7 is that the system must identify the location of connector 207 or rotate the connector 204 such that the connector 207 can be in a dedicated position for making a connection for pressure measurement. Instead, the electrical signals from wires 206 can be guided to a plurality of conductive rings. FIG. 8 shows an exemplary arrangement of the conductive rings 210 which are positioned along a shared axis. The shared axis can be the rotation axis of the optical connector 204. The orientation of the connector 204 does not affect making contact to these rings, which simplifies connection arrangement for the pressure measurement. Note that the relative position of each ring along the axis can be flexible. For instance, rings of different diameters can be arranged such that they overlap in this cut view in FIG. 8.

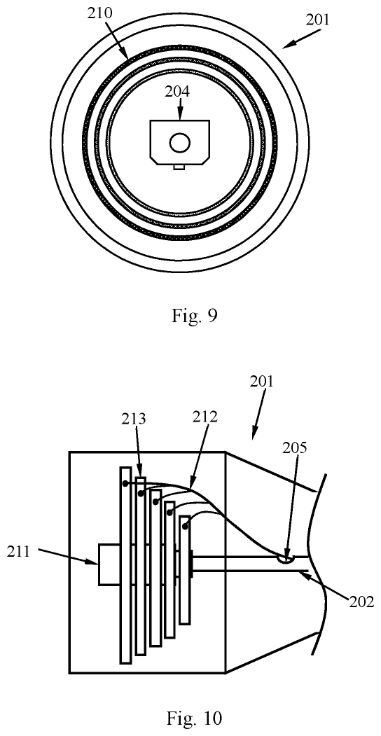

FIG. 9 shows the end view of the design in FIG. 8, which shows that the conductive rings are arranged in a concentric manner in this view.

FIG. 10 shows another exemplary embodiment of the proximal mating unit 201, which can be used for imaging and pressure measurement combination probes where both channels are electrical. The electrical signals from wires 212 can be guided to a plurality of conductive rings 213 which are positioned along a shared axis. The shared axis can be the rotation axis of the longitudinal tube 202, which provides the torque to rotate the transducers at the distal end of the probe. A mechanical connector 212 can be used to rotate the tube 202.

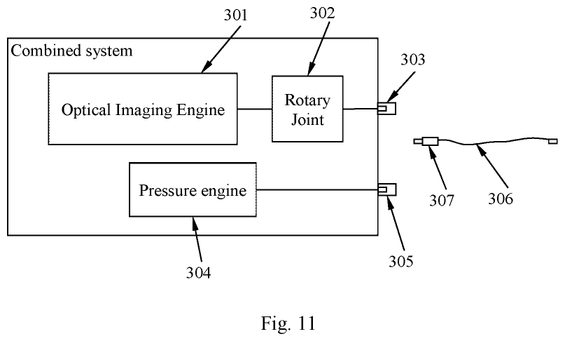

FIG. 11 shows an exemplary embodiment of the combined optical imaging/pressure measurement engine, which includes an optical imaging engine 301, an optical rotary joint 302, a mating sleeve for optical imaging 303, a pressure measurement engine 304 and a mating sleeve for pressure measurement 305. The combined optical imaging/pressure measurement probe 306 can be mated to either mating sleeve via the proximal mating unit 307.

1. A combination system for an optical imaging apparatus and/or a pressure measurement apparatus comprising:
a) a combination optical or ultrasound imaging and pressure measurement probe having at least one well defined longitudinal bore which includes a rotatable arrangement encompassing at least a portion of a first signal channel and a portion of a second signal channel;
b) an optical or ultrasound imaging apparatus in communication with the said first signal channel, which includes a first optical or electrical signal transmission line;
c) a pressure measurement apparatus in communication with the said second signal channel which includes a pressure transducer.

2. The system of claim 1, wherein a portion of the combination probe which encompasses the said first signal channel and the said second signal channel has a size of less than 3 french.

3. The system of claim 1, wherein the first signal channel and the second signal channel are enclosed in a rotation transfer device.

4. The system of claim 3 wherein the rotation transfer device is a torque coil.

5. The system of claim 1, wherein the said bore defines at least a first opening to the environment from which the pressure measurement is acquired.

6. The system of claim 1, wherein the said pressure transducer is encompassed by a protective sheath inside the said bore.

7. The system of claim 6, wherein the said protective sheath defines at least a second opening via which the pressure in the said bore is transmitted to the pressure transducer.

8. The system of claim 1, wherein the said optical imaging apparatus is configured to perform Optical Coherence Tomography.

9. The system of claim 1, wherein the said optical imaging apparatus is configured to perform spectroscopy.

10. The system of claim 1, wherein the second signal channel transmits the pressure signal optically and includes a second optical fiber.

11. The system of claim 1, wherein the second signal channel transmits the pressure signal electrically and includes a plurality of electrical wires.

12. The system of claim 1, wherein the said second signal channel is terminated with a plurality of conductive rings whose axes are substantially aligned with the axis of the said rotatable arrangement.

13. The system of claim 1, wherein the said first optical fiber is terminated with an optical connector on one end.

14. The system of claim 13, wherein
a) the said optical imaging apparatus includes a fiber optical rotary joint.
b) the said optical connector is connected to the said optical rotary joint.

15. The system of claim 13, wherein the said rotatable arrangement is attached to one end of the said optical connector.
